(19) United States
(12) Patent Application Publication
(10) Pub. No.: US 2015/0164343 A1
(43) Pub. Date: Jun. 18, 2015

(54) MANAGEMENT SYSTEMS AND METHODS FOR MANAGING PHYSIOLOGY DATA MEASUREMENT

(71) Applicant: Industrial Technology Research Institute, Hsinchu (TW)

(72) Inventors: Ching-Yu Huang, Taoyuan County (TW); Szu-Han Tzao, Taipei City (TW); Te-San Liao, Taipei City (TW); Jung-Ping Chen, New Taipei City (TW)

(73) Assignee: Industrial Technology Research Institute, Hsinchu (TW)

(21) Appl. No.: 14/140,401

(22) Filed: Dec. 24, 2013

(30) Foreign Application Priority Data
Dec. 13, 2013 (TW) 102146033

(51) Int. Cl. A61B 5/0205; A61B 5/00; A61B 5/021; A61B 5/145; A61B 5/024

(52) U.S. Cl. CPC A61B 5/02055; A61B 5/14532; A61B 5/024; A61B 5/021; A61B 5/7271

(57) ABSTRACT

Management systems and methods for managing physiology data measurement are provided. First, physiology data input is received. Next, a measurement schedule is obtained according to the physiology data input, wherein the measurement schedule includes a measurement frequency and at least one measurement time point corresponding thereto. Thereafter, at the measurement time point, physiology data measurement is performed to obtain a measured value. The measurement frequency and/or the measurement time point of the measurement schedule is dynamically updated based on the measured value and a predefined abnormality determination criterion and subsequent measurements are to be performed with the updated measurement frequency and/or the measurement time point.

### CROSS REFERENCE TO RELATED APPLICATIONS

This application claims priority of Taiwan Patent Application No. 102146033, filed on Dec. 13, 2013, and the entirety of which is incorporated by reference herein.

### TECHNICAL FIELD

The application generally relates to management systems and methods for managing physiology data measurement capable of dynamically adjusting measurement frequency and measurement time points.

### BACKGROUND

In recent years, as society ages and the prevalence of chronic disease increases, a long-term conservation track to analyze and judge patients' physiological conditions has become more and more important. A patient may use a physiological measurement device, such as a blood glucose meter, to perform a medical test on their own to obtain measurement results for physiology data such as blood glucose value, and record the measured values of the physiological measurements in order for health care specialists for interpretation and determine subsequent treatments accordingly.

Taking diabetes care as an example, medical experts should employ blood glucose values measured by patients themselves at assigned measurement time points and measurement frequencies (such as performing the measurement twice before and after eating breakfast every week) through correctly timing (as before or after eating) to analyze long-term trends of interpretation to derive a better understanding of the physiological status of the patient with regards to blood glucose aspects, so as to recommend follow-up treatments and to more precisely, and in real-time, handle the prescription of the most appropriate medication.

However, currently used blood glucose measurements with fixed measurement frequency and measurement time points cannot provide a patient with enough information about the changes in blood glucose levels, which can lead to medical experts being unable to effectively analyze and interpret the long-term trends of the changes in blood glucose, and thus they cannot effectively provide recommendations on follow-up treatment. In addition, if an unnecessary blood glucose measurement is performed frequently, the measurement costs may increase, thereby reducing the performance and intention of patient self-management.

It is therefore desirable to provide methods and systems for efficiently managing physiology data measurement.

### SUMMARY

Management methods and systems for managing physiology data measurement are provided.

In accordance with the application an exemplary embodiment of a management method for managing physiology data measurement is provided. First, physiology data input is received. Next, a measurement schedule is obtained according to the physiology data input, wherein the measurement schedule includes a measurement frequency and at least one measurement time point corresponding thereto. Thereafter, at the measurement time point, physiology data measurement is performed to obtain a measured value. The measurement frequency and/or the measurement time point of the measurement schedule is dynamically updated based on the measured value and a predefined abnormality determination criterion and subsequent measurements are to be performed with the updated measurement frequency and/or the measurement time point.

In accordance with the application an exemplary embodiment of a management system for managing physiology data measurement is provided, wherein the management system comprises an input unit, a storage unit, and a physiology data analyzing unit. The input unit receives physiology data input. The storage unit stores a database for storing the physiology data input. The physiology data analyzing unit, which is coupled to the input unit and the storage unit, obtains a measurement schedule according to the physiology data input, wherein the measurement schedule includes a measurement frequency and at least one measurement time point corresponding thereto, performs a physiology data measurement to obtain a measured value at the measurement time point, and dynamically updates the measurement frequency and/or the measurement time point of the measurement schedule based on the measured value and a predefined abnormality determination criterion and performs subsequent measurements with the updated measurement frequency and/or the updated measurement time point.

The principles of aspects and features of the application will become apparent to those with ordinary skill in the art upon review of the following descriptions of specific embodiments of the management methods and systems for managing physiology data measurement.

### BRIEF DESCRIPTION OF DRAWINGS

The application can be more fully understood by reading the subsequent detailed description and exemplary embodiments with references made to the accompanying drawings, wherein:

FIG. 1 is a block diagram illustrating a management system for managing physiology data measurement according to an exemplary embodiment of the application;

FIG. 2 is a flow chart illustrating a management method for managing physiology data measurement according to an exemplary embodiment of the application;

FIG. 3 is a flow chart illustrating a management method for managing physiology data measurement according to another exemplary embodiment of the application;

FIG. 4 shows a schematic diagram illustrating an embodiment of the table for risk level estimation according to the application;

FIG. 5 shows a schematic diagram illustrating an exemplary embodiment of the abnormal probability array according to the application; and

FIGS. 6A and 6B shows a schematic diagram illustrating an exemplary embodiment of the predefined abnormality determination criterion records according to the application.

### DETAILED DESCRIPTION OF THE EMBODIMENTS

This description is made for the purpose of illustrating the general principles of the application and exemplary embodiments will be described in detail with reference to accompanying drawings so as to be easily realized by a person having ordinary knowledge in the art. The inventive concept may be embodied in various forms without being limited to the exemplary embodiments set forth herein.

Embodiments of the application provide methods and systems for blood glucose measurement capable of dynamically adjusting and scheduling the measurement of the blood glucose, which can dynamically plan/schedule the time point at which a user should perform the blood glucose measurement according to statistics analyzing results of the blood control performance with time for that user and then automatically prompt the user to perform the measurement when the planned/scheduled time point is reached.

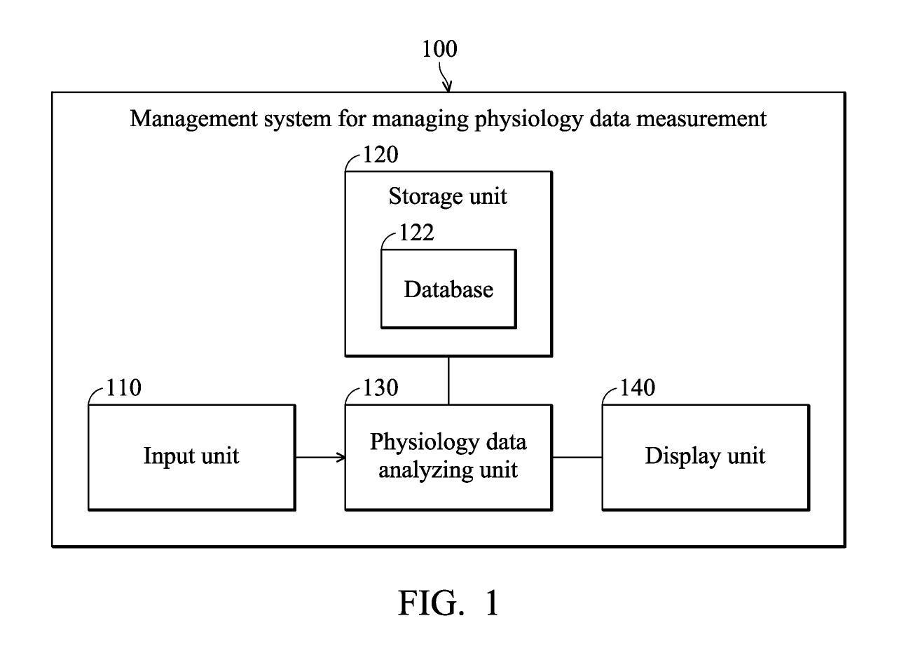

FIG. 1 is a block diagram illustrating a management system for managing physiology data measurement according to an exemplary embodiment of the application.

The management system for managing physiology data measurement 100 can be used in an electronic device, such as a blood glucose meter, a blood pressure monitoring system, a PDA (Personal Digital Assistant), a smartphone, a mobile phone, an MID (Mobile Internet Device, MID), a laptop computer, a car computer, a digital camera, a multi-media player, a gaming device, or any other type of mobile computational device, however it is to be understood that the disclosure is not limited thereto. The management system for managing physiology data measurement 100 at least comprises an input unit 110, a storage unit 120 and a physiology data analyzing unit 130. The input unit 110, the storage unit 120 and the physiology data analyzing unit 130 can be implemented by suitable hardware, software or by a combination of the hardware, software and firmware. The input unit 110 may receive physiology data input. Items of the physiology data input can include basic data of a user, such as age, sex, case history and other personal data of the user and/or measurement results of various physiology data such as blood glucose data, blood pressure data, pulse data, body temperature data and weight data. It is understood that, the physiology data may be manually input by the user or it may be the physiology data measurement result (e.g. the blood glucose value) which is measured by the physiology data measurement device (e.g. the blood glucose meter) and output to the input unit 110 automatically after the physiology data measurement device performs a measurement. The physiology data measurement device can be an external device connected to the management system for managing physiology data measurement 100 or it can be built-in within the management system for managing physiology data measurement 100 for performing a measurement with specific physiology data (e.g. the blood glucose data or the blood pressure data). For example, the physiology data measurement device can be an external blood glucose meter which is coupled to the input unit 110 of the management system for managing physiology data measurement 100 for measuring the blood glucose value of the user to obtain a measured value of the blood glucose and then automatically outputting the measured value of the blood glucose to the input unit 110.

The storage unit 120 (e.g. a built-in memory, hard disk or an external memory card or other storage device) which stores related data, such as a plurality of different user data and respective measured values of physiology data. The storage unit 120 further stores a database 122 for storing the basic data and respective measured values of physiology data of multiple users and may include previous measurement logs of physiology data and variation modes. In addition, the database 122 may further store extra auxiliary information of the user, such as information regarding food and drink, sport, sleep and so on.

The management system for managing physiology data measurement 100 may further comprise a display unit 140 (e.g. a LCD display device). The display unit 140 can display related data, such as texts, figures, interfaces, and/or information. It is understood that, in some embodiments, the display unit 140 may be integrated with a touch-sensitive device (not shown). The touch-sensitive device has a touch-sensitive surface comprising sensors in at least one dimension to detect contact and movement of at least one object (input tool), such as a pen/stylus or finger near or on the touch-sensitive surface. Accordingly, users are able to input commands or physiology data via the display unit 140.

When the user inputs the physiology data via the display unit 140, the physiology data analyzing unit 130 may store the physiology data input in the database 122 for subsequent use. The physiology data analyzing unit 130 (e.g. a processor or microprocessor) can perform the management method for managing physiology data measurement of the application to dynamically adjust the measurement frequency and measurement time points, which will be discussed further in the following paragraphs. To be more specific, the physiology data analyzing unit 130 may perform a risk level classification according to the measured value of physiology data received from the input unit 110, refer to the previous physiology data and variation mode of the user recorded in the database 122 to adjust the measurement frequency and scheduled measurement time points and then dynamically plan the measurement frequency and measurement time points according to the blood control performance with time for the user. The physiology data analyzing unit 130 may also automatically prompt the user to perform the blood glucose measurement when the planned/scheduled time point is reached. Note that the measurement frequency can be represented by the measurement time period and the number of measurements. For example, the measurement frequency can be set as “the measurement is performed at least one time every week”, “the measurement is performed at least one time every day”, “the measurement is performed at least a number of times every week or every day” and so on, and the disclosure is not limited thereto. The scheduled measurement time point may include different life behavior periods, such as “when empty stomach”, “at breakfast”, “after breakfast and before lunch”, “at lunch”, “after lunch and before dinner”, “at dinner”, “after dinner and before bedtime” or other time periods, or it may in a time unit of a hour, a day, a week or a month, or it may be a combination thereof. For example, in one embodiment, the measurement frequency can be set as measuring seven times every day and the scheduled measurement time points can be a total of seven measurement time points, which are “before eating breakfast”, “after eating breakfast”, “before eating lunch”, “after eating lunch”, “before eating dinner”, “after eating dinner” and “at bedtime”, which means that the user should perform the blood glucose measurement at those seven measurement time points. However, it is to be understood that the disclosure is not limited thereto.

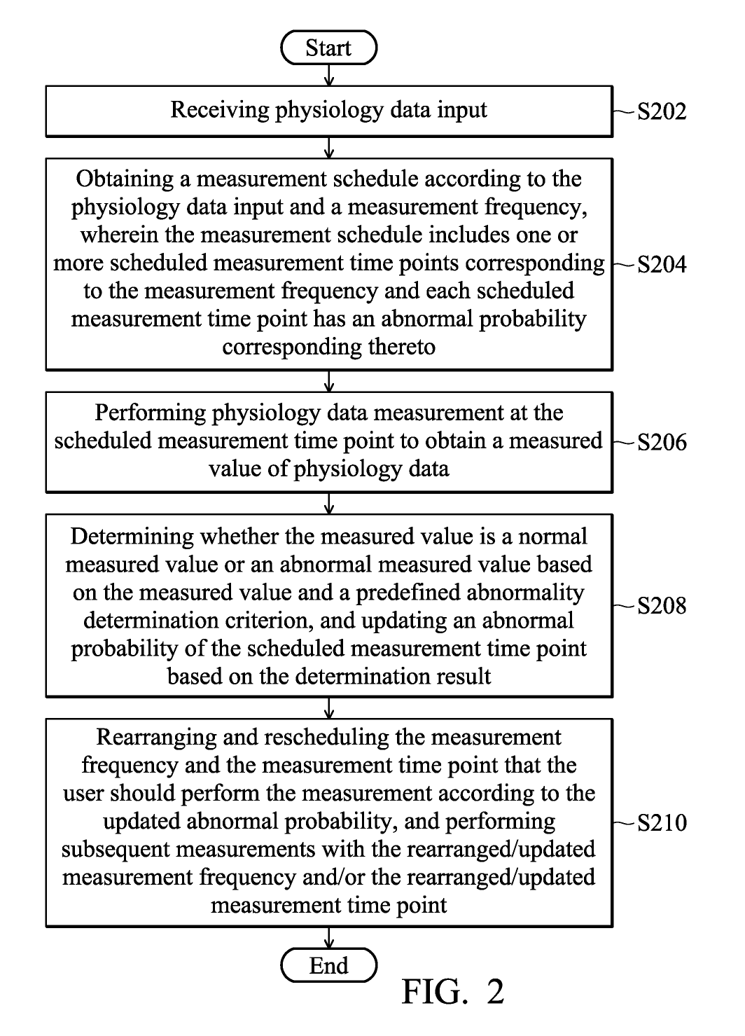

FIG. 2 is a flow chart illustrating a management method for managing physiology data measurement according to an exemplary embodiment of the application. In this embodiment, the management method for managing physiology data measurement can be applied in the management system for managing physiology data measurement 100 as shown in FIG. 1.

First, in step S202, the physiology data analyzing unit 130 receives physiology data input via the input unit 110. For example, items of the physiology data input can include basic data of a user, such as age, sex, case history (e.g. the abnormal mode) and other personal data of the user and/or measurement results of various physiology data such as blood glucose data, blood pressure data, pulse data, body temperature data and weight data. The physiology data may be manually input by the user or it may be the physiology data measurement result (e.g. the blood glucose value) which is measured by the physiology data measurement device (e.g. the blood glucose meter) and output to the input unit 110 automatically after the physiology data measurement device performs a measurement.

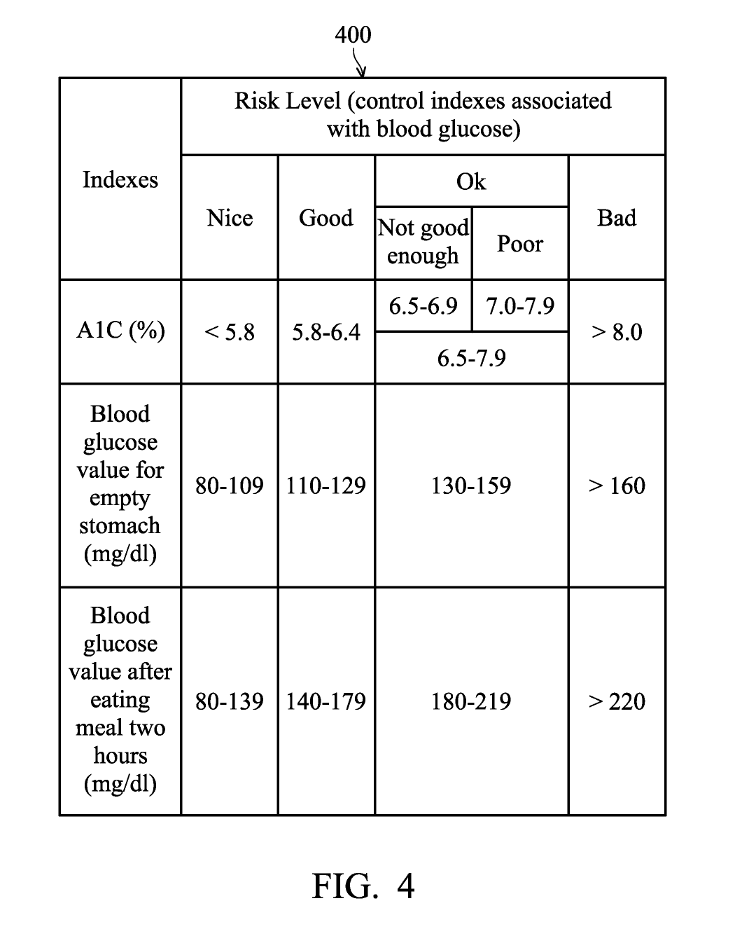

Next, in step S204, the physiology data analyzing unit 130 obtains a measurement schedule according to the physiology data input and an initial measurement frequency, wherein the measurement schedule includes one or more measurement time points corresponding to the initial measurement frequency. Note that the measurement frequency can be represented by the measurement time period and the number of measurements. For example, the measurement frequency can be set as “the measurement is performed at least one time every week”, “the measurement is performed at least one time every day” or “the measurement is performed at least a number of times every week or every day” and so on, and the disclosure is not limited thereto. The initial measurement frequency may be determined according to the measured value of physiology data and a risk level estimation table. The risk level estimation table has defined a number of reference indexes and respective risk levels for representing the control performance of the physiology data (e.g. the blood glucose data), and the physiology data analyzing unit 130 may perform the risk level estimation for each physiology data based on a risk level estimation table corresponding thereto, thereby obtaining a respective initial measurement frequency accordingly. For example, refer to FIG. 4, FIG. 4 shows a schematic diagram illustrating an embodiment of a risk level estimation table 400 according to the application. The risk level estimation table 400 can be pre-stored in the database 122 for performing the risk level estimation. In this embodiment, as shown in FIG. 4, it is assumed that the risk level estimation table 400 is a medical guideline associated with the blood glucose and can be divided into four levels: “Nice” (Level01), “Good” (Level02), “Ok” (Level03) and “Bad” (Level04), wherein the initial measurement frequencies for these four levels are set as “the measurement is performed at least more than one time every week”, “the measurement is performed at least more than one time every day”, “the measurement is performed at least more than two times every day” and “the measurement is performed at least more than three or four times every day”, respectively. In other words, the initial measurement frequency is set as “the measurement is performed at least more than one time every week” if the risk level is estimated as “Nice” (e.g. the blood glucose value for empty stomach is a value between 90 mg/dl and 139 mg/dl) and it is set as “the measurement is performed at least more than three times every day” if the risk level is estimated as “Bad” (e.g. the blood glucose value for empty stomach is a value larger than 160 mg/dl) and so forth.

The scheduled measurement time point may include different life behavior periods, such as “when empty stomach”, “before breakfast”, “at breakfast”, “after breakfast and before lunch”, “at lunch”, “after lunch and before dinner”, “at dinner”, “after dinner and before bedtime” or other time periods, or it may in a time unit of a hour, a day, a week or a month, or it may be a combination thereof. For example, in one embodiment, the measurement frequency can be set as measuring seven times every day and the scheduled measurement time points can be a total of seven measurement time points, which are “before eating breakfast”, “after eating breakfast”, “before eating lunch”, “after eating lunch”, “before eating dinner”, “after eating dinner” and “at bedtime”, which means that the user should perform the blood glucose measurement at those seven measurement time points. However, it is to be understood that the disclosure is not limited thereto.

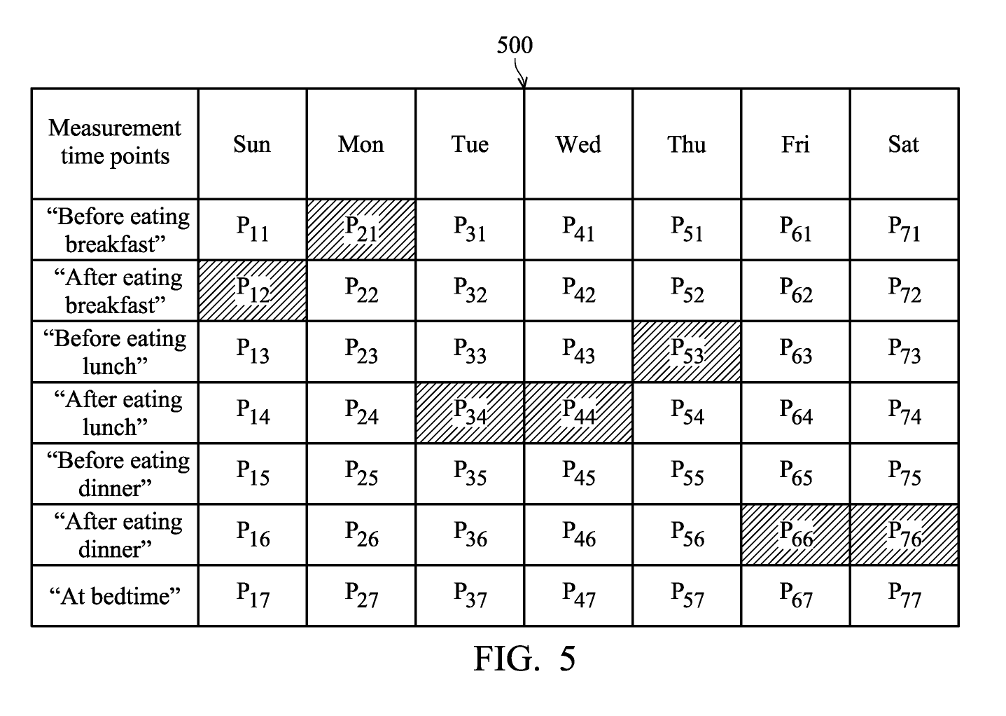

In this embodiment, one or more scheduled measurement time points can be scheduled according to statistically or historically abnormal probabilities of all of possible measurement time points recorded in a corresponding abnormal probability array. Note that the initial abnormal probability array may include the respective abnormal probability for each possible measurement time point during every time period such as every day or every week, wherein the abnormal probability for a specific measurement time point represents the probability that the measured value of physiology data is abnormal at the specific measurement time point. In other words, each of the scheduled measurement time points has a corresponding abnormal probability in the abnormal probability array. FIG. 5 shows a schematic diagram illustrating an exemplary embodiment of the abnormal probability array according to the application. The abnormal probability array 500 in this embodiment can be pre-stored in the database 122 for providing abnormal probabilities corresponding to all possible measurement points within a week. As shown in FIG. 5, the abnormal probability array 500 may include possible measurement points and associated statistically abnormal probabilities, wherein the abnormal probability values P₁₁ to P₇₇ represent the abnormal probabilities corresponding to all possible measurement points within a week. For example, P₁₁ represents the abnormal probability for the measurement point “before eating breakfast on Sunday”, P₂₁ represents the abnormal probability for the measurement point “before eating breakfast on Monday”, and so forth. In one embodiment, when the database has stored a user log corresponding to the basic data of the user (i.e. the user is an old user), the physiology data analyzing unit 130 may find the initial abnormal probability array corresponding to the user from the database directly. In another embodiment, when the database does not store any user log corresponding to the basic data of the user (i.e. the user is a new user), the physiology data analyzing unit 130 may perform case analysis and comparison on the user logs in the database 122 by multidimensional scaling to obtain a user group with a plurality of similar user logs similar to that of the user from the database 122 using the basic data of the user. After that, the physiology data analyzing unit 130 may estimate a probability distribution for the occurrence of the abnormal value within a week based on history data associated with the found user group and determine an initial measurement schedule according thereto. For example, the database 122 has pre-stored information regarding multiple users and their respective abnormal probability arrays, and when the user is a level one diabetic patient with an age of 50 years old and a sex of male, the physiology data analyzing unit 130 may first find out N (e.g. three) similar user logs with age and case history similar to that of the user from the database 122 and then perform a mathematical operation, such as the average operation and/or the weight operation, on the abnormal probabilities of all the measurement points in the probability arrays of the N similar user logs to calculate an initial probability array corresponding to the user. In this embodiment, the physiology data analyzing unit 130 may choose the possible measurement point with the highest abnormal probability among all of the possible measurement points within in a day in the initial probability array to be the scheduled measurement time point, but the disclosure is not limited thereto.

After the initial probability array corresponding to the user has determined, in step S206, the physiology data analyzing unit 130 performs physiology data measurement at the scheduled measurement time point to obtain a measured value of physiology data. For example, when the physiology data is set to be the blood glucose data and the scheduled measurement time points are set to be “before and after eating breakfast on Sunday”, the physiology data analyzing unit 130 will perform or prompt the user to perform the blood glucose measurements before and after eating breakfast on Sunday separately and obtain the respective measured values of physiology data at these time.

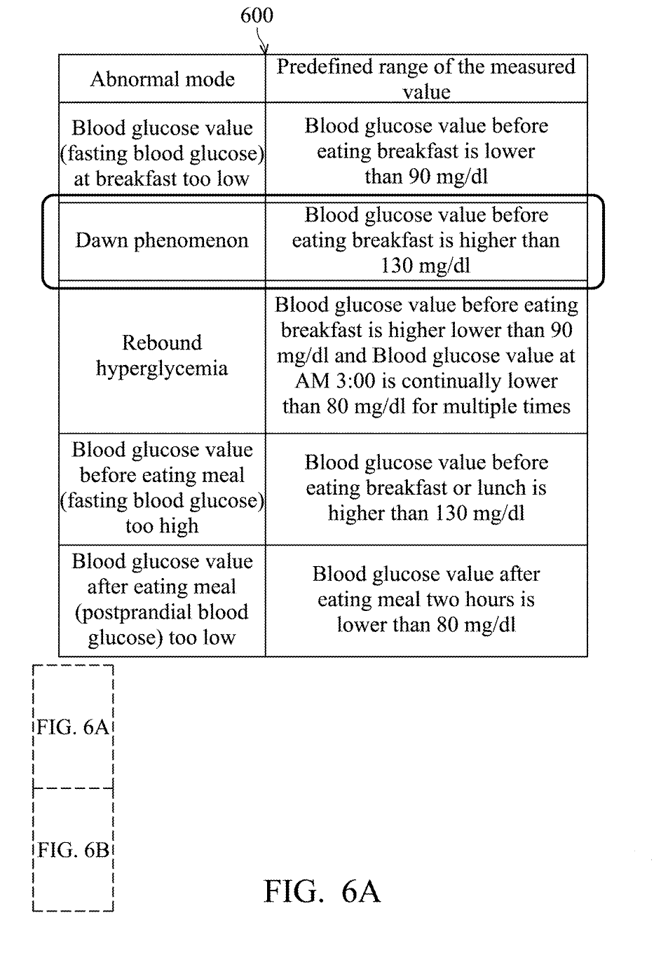

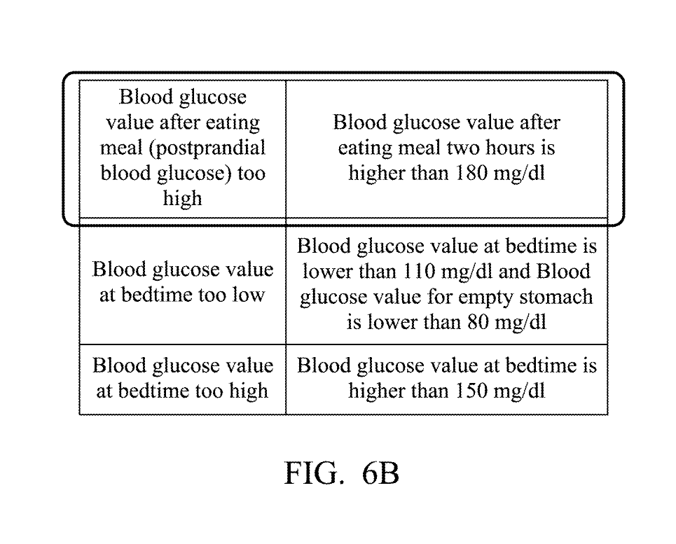

After obtaining the measured value of physiology data, in step S208, the physiology data analyzing unit 130 further determines whether the measured value is a normal measured value or an abnormal measured value based on the measured value and a predefined abnormality determination criterion, and updates an abnormal probability of the scheduled measurement time point based on the determination result. The predefined abnormality determination criterion includes measurement time points and respective predefined ranges of measured value for the measurement time points. When the measured value of a specific measurement point is in the predefined range of a measured value corresponding to the specific measurement point, the physiology data analyzing unit 130 determines that the measured value is the normal measured value; otherwise, determines that the measured value is the abnormal measured value. FIGS. 6A and 6B show a schematic diagram illustrating an exemplary embodiment of the predefined abnormality determination criterion record according to the application. The predefined abnormality determination criterion record 600 in this embodiment can be pre-stored in the database 122 for providing information to determine whether the measured value is the normal measured value or the abnormal measured value. As shown in FIGS. 6A and 6B, the predefined abnormality determination criterion record 600 may include predefined ranges of measured value for different measurement time points and the physiology data analyzing unit 130 may obtain a predefined range of the measured value corresponding to each measurement time point from the predefined abnormality determination criterion record 600. For example, referring to FIGS. 6A and 6B, if the measurement time point is set as the time point “before breakfast”, the predefined range of the measured value for this measurement time point is that the measured value is less than or equal to 130 mg/dl. Furthermore, the physiology data analyzing unit 130 may further find out an abnormal mode for the abnormal measured value from the predefined abnormality determination criterion record 600, wherein the abnormal mode may further be utilized to determine suitable measurement time points. It is understood that, although FIGS. 6A and 6B are taken the predefined abnormality determination criterion record for the blood glucose measurement as an example for illustration, but the disclosure is not limited thereto. In other words, different predefined abnormality determination criterion record can be applied to different physiology data and thus the application can also be applied to abnormal determinations for a variety of physiology data.

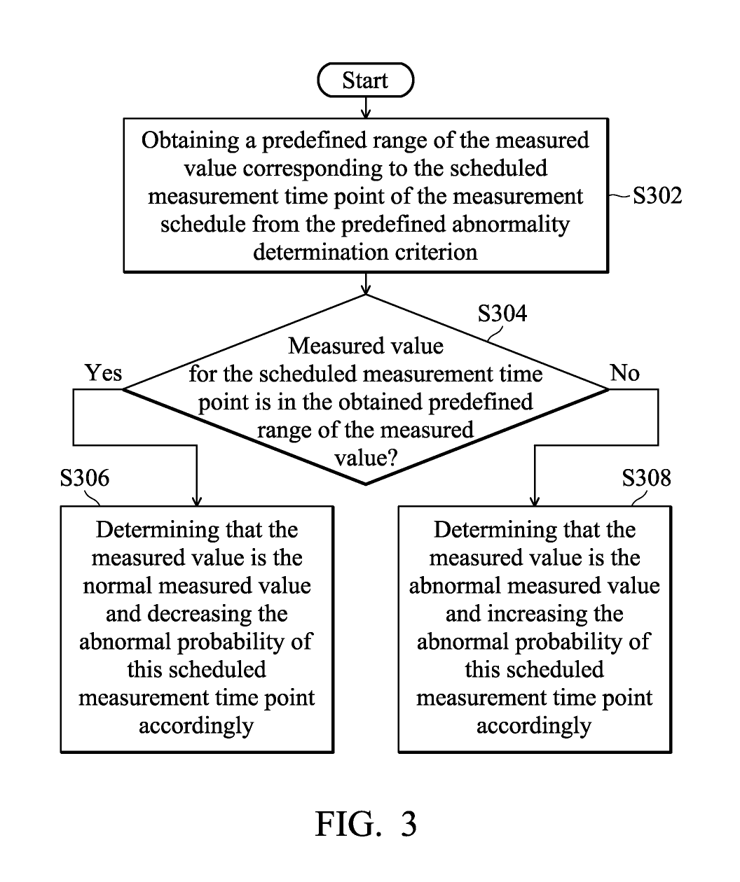

After determining that the measured value is the normal measured value or the abnormal measured value, the physiology data analyzing unit 130 further updates the abnormal probability corresponding to the scheduled measurement time point in the abnormal probability array. To further clarify, when the measured value of a first measurement time point is determined as the normal measured value, this means that the measured value is normal in this measurement, the physiology data analyzing unit 130 decreases the abnormal probability of the first measurement time point. Contrarily, when the measured value of a second measurement time point is determined as the abnormal measured value, which means that the measured value is abnormal at the second measurement time point, the physiology data analyzing unit 130 increases the abnormal probability of the second measurement time point. Determination of the abnormal measured value and updating of the abnormal probability will be described in detail with reference to FIG. 3.

After determining that the measured value is the normal measured value or the abnormal measured value and updating the abnormal probability corresponding to the scheduled measurement time point in the abnormal probability array accordingly, in step S210, the physiology data analyzing unit 130 rearranges and reschedules the measurement frequency and the measurement time point that the user should perform the measurement according to the updated abnormal probability, and performs subsequent measurements with the rearranged/updated measurement frequency and/or the rearranged/updated measurement time point. Thus, the physiology data analyzing unit 130 can dynamically adjust the measurement frequency and the measurement time points for each day or week based on actual abnormal probabilities so that the time when the abnormal is occurred can easily be detected, making reading of the doctor easily.

Thereafter, the physiology data analyzing unit 130 may further display a prompting signal on the display unit 140 to prompt the user via text, picture, voice or other manners to perform the physiology data measurement when the scheduled measurement time point is reached.

FIG. 3 is a flow chart illustrating a management method for managing physiology data measurement according to another exemplary embodiment of the application. In this embodiment, the management method for managing physiology data measurement can be applied in the management system for managing physiology data measurement 100 as shown in FIG. 1 for updating the abnormal probability corresponding to the scheduled measurement time point in the abnormal probability array.

First, the physiology data analyzing unit 130 obtaining a predefined range of the measured value corresponding to the scheduled measurement time point of the measurement schedule from the predefined abnormality determination criterion (step S302). For example, referring to FIGS. 6A and 6B, if the scheduled measurement time point is set as the time point “before breakfast”, the predefined range of the measured value for this measurement time point is that the measured value is less than or equal to 130 mg/dl.

Thereafter, the physiology data analyzing unit 130 further determines whether the measured value for the scheduled measurement time point is in the obtained predefined range of the measured value (step S304). As in previously described example, determination of whether the measured value for the scheduled measurement time point is in the obtained predefined range of measured is to determine whether the measured value is less than or equal to 130 mg/dl.

In response to determining that the measured value is in the obtained predefined range of the measured value (Yes in step S304), e.g. the measured value is 120 mg/dl, the physiology data analyzing unit 130 determines that the measured value is the normal measured value and thus decreases the abnormal probability of this scheduled measurement time point and updates the probability distribution of occurrence of abnormal point for that day accordingly (step S306). For example, but not limited to, in one embodiment, if original abnormal probability P_{ij(old)} is set to P_{ij(old)}=uᵢⱼ/dᵢⱼ, the abnormal probability of the scheduled measurement time point can be decreased to obtain the updated abnormal probability P_{i,j(new)} by the following equation:

$${P}_{ij ( new )} = \frac{{u}_{ij}}{{d}_{ij} + l} ,$$

where l represents a constant value relative to the measurement frequency.

Contrarily, in response to determining that the measured value is not in the obtained predefined range of the measured value (No in step S304), e.g. the measured value is 140 mg/dl, the physiology data analyzing unit 130 determines that the measured value is the abnormal measured value and thus increases the abnormal probability of this scheduled measurement time point and updates the probability distribution of occurrence of abnormal point for that day accordingly (step S308). For example, but not limited to, in one embodiment, if original abnormal probability P_{ij(old)} is set to P_{ij(old)}=uᵢⱼ/dᵢⱼ, the abnormal probability of the scheduled measurement time point can be increased to obtain the updated abnormal probability P_{i,j(new)} by the following equation:

$${P}_{ij ( new )} = \frac{{u}_{ij} + k}{{d}_{ij} + k} ,$$

where k represents a constant value relative to the measurement frequency.

For example, in one embodiment, if the constants l and k are both set to be 1 and the original abnormal probability P_{i,j(old)} is set to P_{i,j(old)}=½, the updated abnormal probability P_{i,j(new)} is set to P_{i,j(new)}=⅓ when the measured value is in the obtained predefined range of the measured value; similarly, the updated abnormal probability P_{i,j(new)} is set to P_{i,j(new)}=⅔ when the measured value is not in the obtained predefined range of the measured value. Therefore, the application can update the historical abnormal probability corresponding to the scheduled measurement time point in the abnormal probability array such that the abnormal measurement time point can easily be selected out for measurement in subsequent processes.

In another embodiment, the abnormal probability of the scheduled measurement time point can be increased by the following equation:

when the abnormal measured value is obtained at the scheduled measurement time point, the probability parameter of occurrence of abnormal point for that day can be updated as below:

$${P}_{ijt} = \frac{{u}_{ij ( t - 1 )} + k ( {d}_{t - 1} , c {0}_{t - 1} )}{{d}_{ij ( t - 1 )} + k ( {d}_{t - 1} , c {0}_{t - 1} )} ,$$

where dₜ₋₁ represents a difference between the measured value Oₜ₋₁ obtained and the standard measured value Sᵢⱼ defined at the time point (t−1): dₜ₋₁=Oₜ₋₁−Sᵢⱼ,

c0ₜ₋₁ represents the number of the abnormal value continually measured until the time point (t−1) and k represents a function of the dₜ₋₁ and c0ₜ₋₁.

when the normal measured value is obtained at the scheduled measurement time point, the abnormal probability of the scheduled measurement time point can be decreased and the probability parameter of occurrence of abnormal point for that day can be updated as below:

$${P}_{ijt} - \frac{{u}_{ij ( t - 1 )}}{{d}_{ij ( t - 1 )} + l ( {C}_{t - 1} , c {1}_{t - 1} )}$$

where c1ₜ₋₁ represents the number of the normal value continually measured until the time point (t−1) and a function of the Oₜ₋₁ and c1ₜ₋₁.

In some embodiments, when the abnormal measured value is obtained at the scheduled measurement time point, the physiology data analyzing unit 130 may further link to other device to obtain to obtain auxiliary information of the user, such as information regarding food and drink, sport, sleep and so on or obtain it from the database 122 directly.

Thereafter, the physiology data analyzing unit 130 may reschedule the measurement frequency and the measurement time point that the user should perform the measurement according to the updated abnormal probability array, perform subsequent measurements with the updated measurement frequency and/or the updated measurement time point and dynamically adjust the measurement schedule every week.

For example, in one embodiment, when the scheduled measurement time points are set to be a total of seven measurement time points, which are “before eating breakfast”, “after eating breakfast”, “before eating lunch”, “after eating lunch”, “before eating dinner”, “after eating dinner” and “at bedtime” for one day and the historical abnormal probability distributions for these seven measurement time points are set to be (5/28, 3/28, 4/28, 4/28, 4/28, 4/28, 4/28), respectively, if the measurement frequency is set as measuring only once every day, as the abnormal probability parameter for the measurement time point “before eating breakfast” has the highest abnormal probability among all of the seven measurement time points, which means that a patient user may have highest probability to obtain the abnormal measured value before eating breakfast (i.e. the patient user may have a so-called dawn phenomenon), the measurement schedule can be set to be “the measurement is perform once before eating breakfast everyday”.

In some embodiments, when the physiology data measurement is set to be the blood glucose measurement, the management method for managing physiology data measurement with the dynamic adjustment capability of the application can be applied to achieve in blood glucose control and diabetes management. First, the physiology data analyzing unit 130 can receive physiology data input including the basic data of a user and the measured value of blood glucose via the input unit 110 and then perform the risk level estimation of the blood glucose control to distinguish the risk level of the blood glucose control among “Nice”, “Good”, “Ok”, “Bad” and so on using a known medical guideline associated with the blood glucose according to the measured value of blood glucose and value of Glycohemoglobin (also referred to as HbA1C or A1C) blood glucose test for Diabetes.

Thereafter, the physiology data analyzing unit 130 may suggest suitable measurement frequency and scheduled measurement time points according to the basic data of the user and result of the risk level estimation, wherein the scheduled measurement time points sampled in each round can be, for example, a total of seven measurement time points, which are “before eating breakfast”, “after eating breakfast”, “before eating lunch”, “after eating lunch”, “before eating dinner”, “after eating dinner” and “at bedtime”, but it is not limited thereto.

The physiology data analyzing unit 130 may recursively feedback the measured data to calculate and adjust estimated risk level dynamically and based on the position of the abnormal measurement point having abnormal measured value of blood glucose in previous round, increase the number of measurements performed at that measurement point in current loop.

The physiology data analyzing unit 130 may then provide a personal-optimized measurement schedule for blood glucose measurement which is built according to the basic data of the user and continually measurement results, wherein the personal-optimized measurement schedule includes suggested measurement frequency and measurement time points to perform the measurement. Thereafter, the physiology data analyzing unit 130 may further display a prompting signal on the display unit 140 via text, picture, voice or other manners to prompt the user to perform long time physiology data measurement when each scheduled measurement time point is reached.

Accordingly, the patient user may later provide the record of blood glucose measurement to the doctor for subsequent diagnosis when back to hospital. The physiology data analyzing unit 130 may re-suggest suitable measurement frequency and measurement time points to perform the measurement after obtaining a different risk level of blood glucose control which is an estimated risk level determined by the doctor according to the measured values of blood glucose and the value of Glycohemoglobin (HbA1c, A1c) blood glucose test for Diabetes, thus achieving in efficiently blood glucose self-monitoring and diabetes management.

Therefore, according to management methods and systems for managing physiology data measurement capable of dynamically adjusting and scheduling the measurement of the application, in a given measurement frequency, the measurement frequency and scheduled measurement time points for physiology data measurement can be dynamically scheduled according to the blood glucose behavior mode analyzed and learned based on previous measurement results to effectively and economically obtain useful variation message of physiology data such that the medical professional can quickly interpret and determine suitable subsequent treatment for the patient according thereto. Moreover, the management methods and systems for managing physiology data measurement with dynamically adjustment capability of the application can further prompt the user to perform long time physiology data measurement according to the scheduled measurement points, thereby efficiently saving measurement cost and increasing the performance and intention for self-management of users.

The methods may be implemented in program code stored in a machine-readable storage medium, such as a magnetic tape, semiconductor, magnetic disk, optical disc (e.g., CD-ROM, DVD-ROM, etc.), or others, and when loaded and executed by a processing unit, a micro-control unit (MCU), or the controller module 114 in FIG. 1, the program code may perform the D2D communications method in a D2D communications system. In addition, the method may be applied to any D2D capable mobile communications device supporting the WCDMA technology and/or the LTE technology.

While the application has been described by exemplary embodiments, it is to be understood that the application is not limited thereto. It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A management method for managing physiology data measurement, comprising:
receiving physiology data input;
obtaining a measurement schedule according to the physiology data input, wherein the measurement schedule includes a measurement frequency and at least one measurement time point corresponding thereto;
performing a physiology data measurement to obtain a measured value at the measurement time point; and
dynamically updating the measurement frequency and/or the measurement time point of the measurement schedule based on the measured value and a predefined abnormality determination criterion and performing subsequent measurements with the updated measurement frequency and/or the updated measurement time point.

2. The management method of claim 1, wherein the physiology data input comprises basic data of a user and the step of obtaining the measurement schedule according to the physiology data input further comprises:
performing a similarity comparison to obtain a plurality of similar user logs similar to that of the user from a database using the basic data of the user, wherein each of the similar user logs includes a probability array and each probability array includes abnormal probabilities of possible measurement time points;
performing a mathematical operation on the probability arrays of the similar user logs to calculate an initial probability array corresponding to the physiology data input; and
determining the measurement time point of the measurement schedule according to the initial probability array.

3. The management method of claim 2, wherein the step of determining the at least one measurement time point of the measurement schedule according to the initial probability array further comprises choosing the possible measurement time point with the highest abnormal probability among all of the possible measurement time points within in a specific time period in the initial probability array to be the measurement time point of the measurement schedule.

4. The management method of claim 1, wherein the measurement time point comprises at least one combination of the following life behavior periods: breakfast, lunch, dinner, before eating, after eating and at bedtime.

5. The management method of claim 1, wherein the physiology data input comprises at least one combination of the following data of the user: blood glucose data, blood pressure data, pulse data, body temperature data and weight data.

6. The management method of claim 1, wherein the step of dynamically updating the measurement frequency and/or the measurement time point of the measurement schedule based on the measured value and the predefined abnormality determination criterion further comprises:
determining whether the measured value is a normal measured value or an abnormal measured value based on the measured value and the predefined abnormality determination criterion, and updating an abnormal probability of the measurement time point of the measurement schedule based on the determination result.

7. The management method of claim 6, wherein the step of determining whether the measured value is the normal measured value or the abnormal measured value based on the measured value and the predefined abnormality determination criterion further comprises:
obtaining a predefined range of a measured value corresponding to the measurement time point of the measurement schedule from the predefined abnormality determination criterion;
determining whether the measured value is in the obtained predefined range of the measured value; and
determining that the measured value is the normal measured value and decreasing the abnormal probability of the measurement time point of the measurement schedule in response to determining that the measured value is in the obtained predefined range of the measured value.

8. The management method of claim 7, wherein the step of determining whether the measured value is the normal measured value or the abnormal measured value based on the measured value and the predefined abnormality determination criterion further comprises:
determining that the measured value is the abnormal measured value and increasing the abnormal probability of the measurement time point of the measurement schedule in response to determining that the measured value is not in the obtained predefined range of the measured value.

9. The management method of claim 1, further comprising the step of linking to at least one device to obtain auxiliary information of the user in response to determining that the measured value is the abnormal measured value.

10. The management method of claim 1, wherein the step of performing subsequent measurements with the updated measurement frequency and/or the updated measurement time point further comprises:
utilizing a prompting signal to prompt the user to perform the physiology data measurement at the updated measurement time point.

11. A management system for managing physiology data measurement, comprising:
an input unit, receiving physiology data input;
a storage unit, storing a database for storing the physiology data input; and
a physiology data analyzing unit coupled to the input unit and the storage unit, obtaining a measurement schedule according to the physiology data input, wherein the measurement schedule includes a measurement frequency and at least one measurement time point corresponding thereto, performing a physiology data measurement to obtain a measured value at the measurement time point, and dynamically updating the measurement frequency and/or the measurement time point of the measurement schedule based on the measured value and a predefined abnormality determination criterion and performing subsequent measurements with the updated measurement frequency and/or the updated measurement time point.

12. The management system of claim 11, wherein the physiology data input comprises basic data of a user and the physiology data analyzing unit further performs a similarity comparison to obtain a plurality of similar user logs similar to that of the user from the database using the basic data of the user, wherein each of the similar user logs includes a probability array and each probability array includes abnormal probabilities of possible measurement time points, performs a mathematical operation on the probability arrays of the similar user logs to calculate an initial probability array corresponding to the physiology data input, and determines the measurement time point of the measurement schedule according to the initial probability array.

13. The management system of claim 12, wherein the physiology data analyzing unit further chooses the possible measurement point with the highest abnormal probability among all of the possible measurement points within in a specific time period in the initial probability array to be the measurement time point of the measurement schedule.

14. The management system of claim 11, wherein the measurement time point comprises at least one combination of the following life behavior periods: breakfast, lunch, dinner, before eating, after eating and at bedtime.

15. The management system of claim 11, wherein the physiology data measurement comprises at least one combination of the following measurements of the user: blood glucose measurement, blood pressure measurement, pulse measurement, body temperature measurement and weight measurement.

16. The management system of claim 11, wherein the physiology data analyzing unit further determines whether the measured value is a normal measured value or an abnormal measured value based on the measured value and the predefined abnormality determination criterion and updates an abnormal probability of the measurement time point of the measurement schedule based on the determination result.

17. The management system of claim 16, wherein the physiology data analyzing unit further determines whether the measured value is the normal measured value or the abnormal measured value based on the measured value and the predefined abnormality determination criterion by obtaining a predefined range of the measured value corresponding to the measurement time point of the measurement schedule from the predefined abnormality determination criterion and determining whether the measured value is in the obtained predefined range of the measured value, wherein the physiology data analyzing unit further determines that the measured value is the normal measured value and decreases the abnormal probability of the measurement time point of the measurement schedule in response to determining that the measured value is in the obtained predefined range of the measured value.

18. The management system of claim 17, wherein the physiology data analyzing unit further determines that the measured value is the abnormal measured value and increases the abnormal probability of the measurement time point of the measurement schedule in response to determining that the measured value is not in the obtained predefined range of the measured value.

19. The management system of claim 11, wherein the physiology data analyzing unit further links to at least one device to obtain auxiliary information of the user in response to determining that the measured value is the abnormal measured value.

20. The management system of claim 11, further comprising a display unit coupled to the physiology data analyzing unit, wherein the physiology data analyzing unit further displays a prompting signal via the display unit to prompt the user to perform the physiology data measurement when the updated measurement time point is reached.
